(19) United States
(12) Patent Application Publication
(10) Pub. No.: US 2009/0137871 A1
(43) Pub. Date: May 28, 2009

(54) RADIO OBSERVATION DEVICE

(75) Inventor: Kentaro Matsumoto, Saitama (JP)

Correspondence Address: GREENBLUM & BERNSTEIN, P.L.C., 1950 ROLAND CLARKE PLACE, RESTON, VA, 20191, US

(73) Assignee: HOYA CORPORATION, Tokyo (JP)

(21) Appl. No.: 12/276,515

(22) Filed: Nov. 24, 2008

(30) Foreign Application Priority Data
Nov. 27, 2007 (JP) P2007-305855

(51) Int. Cl. A61B 1/04

(52) U.S. Cl. 600/117

(57) ABSTRACT

A radio observation device which transmits information on observation subjects has a first coil, an oscillator, a first frequency controller, a frequency-measuring device, and a transmitter. The oscillator generates signals flowing through the first coil within a certain range of frequencies. The first frequency controller is connected to the oscillator, and adjusts the oscillating frequency of the signal flowing through the first coil by controlling said oscillator. The frequency-measuring device measures a resonance frequency of a signal flowing through the first coil and a signal flowing through a second coil being provided external to the radio observation device. The transmitter transmits the information on the observation subjects through said first coil by a carrier wave which oscillates at the resonance frequency.

### BACKGROUND OF THE INVENTION

1. Field of the Invention

The present invention relates to a radio observation device which transmits information on a subject of observation by radio.

2. Description of the Related Art

A radio observation device transmits images and sounds to a remote receiver via radio. A capsule endoscope used as a medical device, and a surveillance camera are examples of a radio observation device.

The capsule endoscope transmits image signals regarding an interior of observation subjects to a receiver via radio. The receiver is situated external to the observation subject, and displays an image which is created by decoding the received signals on a display.

The surveillance camera is located in a room or in open air away from a receiver, and transmits images and/or sounds taken from an observation subject to the receiver via radio. The receiver records the received images and sounds, displays them, and produces sounds through speakers.

The frequencies of the radio signals used by the radio observation device are chosen in consideration of the radio frequencies used by nearby electronic devices and the regulations set in the local country or region. If the radio frequency is preset during manufacturing, separate radio observation devices must be manufactured for each radio frequency. Accordingly, the number of variants of the radio observation device will be increasing. There is known a radio observation device which avoids this scenario because it may be used in multiple countries by deciding beforehand the appropriate radio frequency. A radio signal is sent to the radio observation device before usage, such that the device sets its own radio frequency according to the received radio signal. This construction is disclosed in Japanese Unexamined Patent Publications (KOKAI) Nos. 2007-89891 and 2007-89892.

Additionally, Japanese Unexamined Patent Publication (KOKAI) No. 2007-60526 discloses that the construction adjusts its communicating frequency by transmitting certain commands to a wireless card, confirming the response, and measuring the response time from the wireless card.

However, in many cases, for a surveillance camera to remain covert, it must be small. And when multiple surveillance cameras are to be used simultaneously, they should be moderately priced. A capsule endoscope must be small enough to be readily swallowed by a human. In conventional constructions, a radio observation device comprises a receiving antenna and a circuit which receives a signal for setting its transmission frequency, as well as a device which responds to a signal from the receiver. These constructions add to the cost and size of the device.

### SUMMARY OF THE INVENTION

An object of the present invention is to provide a radio observation device capable of setting its transmission frequency without the need for a receiver on board.

A radio observation device which transmits information on observation subjects has a first coil, an oscillator, a first frequency controller, a frequency-measuring device, and a transmitter. The oscillator oscillates signals flowing through the first coil within a certain range of frequencies. The first frequency controller is connected to the oscillator, and adjusts the oscillating frequency of the signal flowing through the first coil by controlling said oscillator. The frequency-measuring device measures the resonance frequency of a signal flowing through the first coil and a signal flowing through a second coil being provided external to the radio observation device. The transmitter transmits the information on the observation subjects through said first coil by a carrier wave which oscillates at the resonance frequency.

### BRIEF DESCRIPTION OF THE DRAWINGS

The objects and advantages of the present invention will be better understood from the following description, with reference to the accompanying drawings in which:

FIG. 1 is a block diagram of a capsule endoscope according to the first embodiment of the present invention;

FIG. 2 is a block diagram of a device provided external to a body to be tested;

FIG. 3 is a block diagram of a capsule endoscope according to the second embodiment of the present invention;

FIG. 4 is a schematic diagram of a capsule endoscope system and a body to be tested according to the third embodiment of the present invention;

FIG. 5 is a schematic diagram of an endoscope system;

FIG. 6 is a cross-sectional view of the distal end of the endoscope and an opening;

FIG. 7 is an end view taken along line VII-VII of FIG. 6;

FIG. 8 is an end view taken along line VIII-VIII of FIG. 6;

FIG. 9 is an end view taken along line IX-IX of FIG. 6;

FIG. 10 is a cross-sectional view of the distal end of the endoscope inserted into the opening, taken along line VI-VI of FIG. 7;

FIG. 11 is a schematic diagram of the distal end of an endoscope according to the fourth embodiment of the present invention;

FIG. 12 is a schematic diagram of an endoscope connected with a battery charger;

FIG. 13 is a schematic diagram of a surveillance camera according to the fifth embodiment of the present invention;

FIG. 14 is a schematic diagram of a surveillance system according to the sixth embodiment of the present invention;

FIG. 15 is a diagram drawn from the ceiling of a room in which a surveillance system is provided; and

FIG. 16 is a flowchart showing a second available frequency notification process.

### DESCRIPTION OF THE PREFERRED EMBODIMENTS

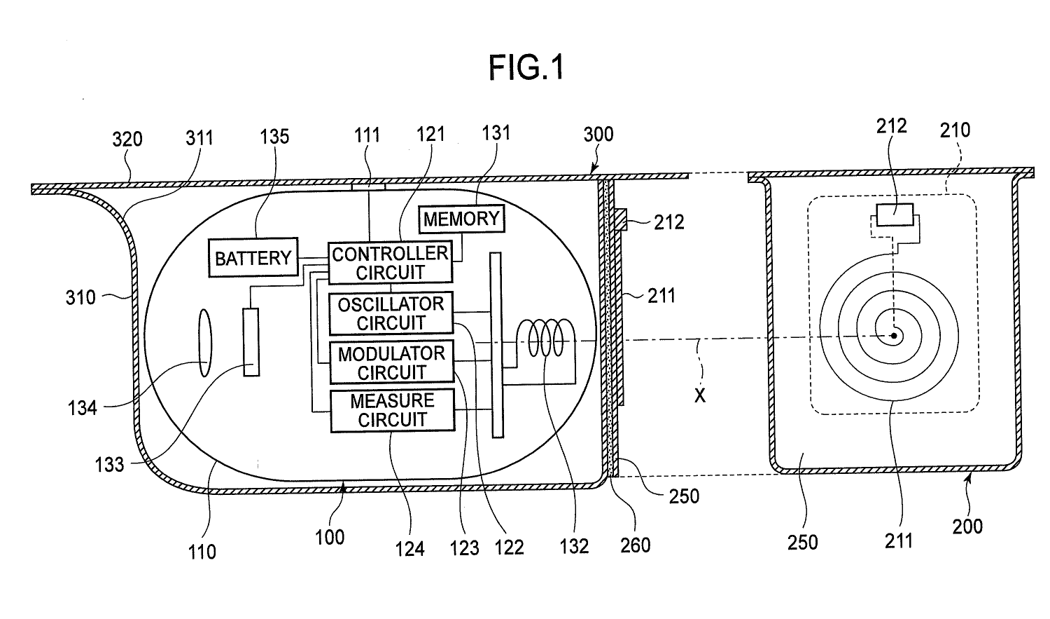

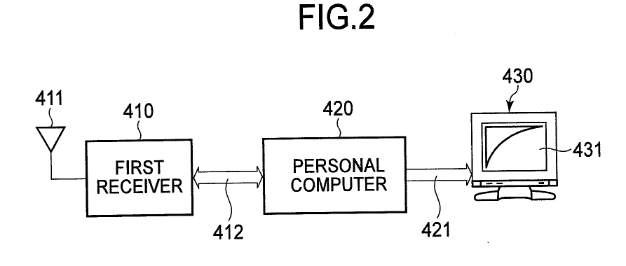

The capsule endoscope 100 and the capsule endoscope system according to the first embodiment of the present invention are described below with reference to FIGS. 1 and 2.

The capsule endoscope 100 mainly comprises a container 110, a power switch 111 which is used to power the capsule endoscope 100, circuits 121, 122, 123, and 124, a memory 131 which stores several pieces of information, a first coil 132 which is used to transmit radio signals, an imaging element (CCD) 133 which images photographic subjects, a lens which provides an image on the CCD 133, and a battery 135 which provides electrical power to the circuits 121, 122, 123, and 124.

The circuits 121, 122, 123, and 124, the first coil 132 behaving as an antenna, the CCD 133, the lens 134, and the battery 135 are stored inside the container 110. The circuits 121, 122, 123, and 124 are composed of a control circuit 121, an oscillator circuit 122, a modulator circuit 123, and a measure circuit 124. The oscillator circuit 122, a modulator circuit 123, and a measure circuit 124 are respectively connected to the control circuit 121 and the first coil 132. The container 110 has a capsule shape which is a cylinder closed at both ends with dome-shaped covers, and has a center axis X which is substantially coaxial with the center axis of the first coil 132.

The first resonator 200 comprises a resonator circuit plate 250, a second coil 211, and a tip capacitor 212 which are provided on the resonator circuit plate 250. A second coil 211 is connected in series with a tip capacitor 212, and forms a first resonance circuit 210 as a serial resonance circuit. Physical values of the second coil 211 and the tip capacitor 212 are selected so that the first resonance circuit 210 will have a particular resonance frequency. That is, any resonance frequency may be configured by selecting the physical values of the second coil 211 and the tip capacitor 212.

The capsule endoscope 100 is stored in the first case 300. The first case 300 comprises a PTP (Press Through Package). The PTP comprises a hard sheet 310 and an aluminum sheet 320. The hard sheet 310 contains a concave portion in which the capsule endoscope 100 is stored. The capsule endoscope 100 is hermetically sealed in the concave portion with the aluminum sheet 320, thereby keeping it in a sterile condition. The resonance circuit plate 250 is attached on the outer surface of the first case 300 with an adhesive sheet, so that the center axis of the second coil 211 is substantially coaxial with the center axis of the first coil 132, and the distance in the axial direction between the first coil 132 and the second coil 211 is in a range that allows electromagnetic induction to occur between the first coil 132 and the second coil 211. The range of electromagnetic induction occurring is decided by electrical power flowing through the first coil 132, the distance between the first coil 132 and the second coil 211, and so on.

The procedure for using the capsule endoscope 100 is described below.

The user pushes the power switch 111 through the aluminum sheet 320 so that the capsule endoscope is powered on. Then, the control circuit 121 reads a control program from the memory 131, executes it, and starts controlling the circuits 121, 122, 123, and 124, the CCD 133, and so on. After that, the control circuit 121 executes a frequency-decision process.

In the frequency-decision process, the oscillator circuit 122 sends a signal of a particular frequency to the first coil 132.

The first circuit 210 is a closed circuit. Therefore, current flows in the first coil 132 so that a magnetic field is created around the first coil 132. The oscillator 122 causes current in the first coil 132 to oscillate at constant frequency, so that the magnetic field changes continually. At the same time, electromagnetic induction occurs in the second coil 211 by a change in the magnetic field around the first coil 132. The electromagnetic induction creates an alternating current in the first resonance circuit.

The oscillator circuit 122 changes the frequency of the signal which is sent to the first coil 132 within a certain range. The frequency of the alternating current flowing through the first resonance circuit 210 corresponds to the resonance frequency of the first resonance circuit 210 as the oscillator circuit 122 changes the frequency of the signal flowing through the first coil 132. Consequently, the first resonance circuit 210 oscillates. The electromagnetic induction created by alternating current in the first resonance circuit generates induced electromotive force in the first coil 132. This induced electromotive force creates current which flows through the first coil 132 in the direction opposite to the current created by the oscillator circuit 122. The current created by the induced electromotive force reduces current flowing through the first coil 132. The frequency of signals sent from the oscillator circuit 122 closely approximates to the resonance frequency of the first resonance circuit 210, when the current flowing through the first coil 132 is minimal.

The measuring circuit 124 measures current flowing through the first coil 132, and send signals to the control circuit 121, while the oscillator circuit 122 varies the frequency. The control circuit 121 adopts the frequency as the DIP frequency when the current is low, and stores the DIP frequency in the memory 131. The DIP frequency is used as the carrier wave frequency for transmitting the image signals. Then, the frequency-decision process ends.

The control circuit 121 starts imaging with the CCD 133, reads the DIP frequency from the memory 131, and sends it to the modulator circuit 123. The modulator circuit 123 receives the image signals from the control circuit 121, and modulates the image signals with the DIP frequency as the carrier wave frequency.

According to these constructions, the capsule endoscope 100 may send radio signals at a frequency which is substantially equal to the resonance frequency of the first resonance circuit 210 without the receiver which a conventional capsule endoscope would comprise.

The user enters the resonance frequency of the first resonance circuit 210 into the first receiver 410. The first receiver receives radio signals from the capsule endoscope 100 at the entered resonance frequency.

The user waits a few seconds until the frequency-decision process ends, then brings out the capsule endoscope 100 from the PTP by pushing the hard sheet 310, and swallows it. The observation thus begins.

The light entering through lens 134 provides an image on the CCD 133. The CCD 133 converts the image to an image signal and sends it to the control circuit 121. The control circuit 121 processes the image signal to create image data. The image data is send to the modulator circuit 123. The modulator circuit 123 modulates the image data with a certain carrier wave frequency sent by the control circuit 121. The modulated data is sent to the first coil 132. The first coil 132 transmits the modulated signals to the outside of the capsule endoscope 100.

The user situates the first receiver 410 external to the body. The first receiver 410 receives radio signals with the first receiver antenna 411, demodulates the received signals, and stores the images created by demodulating the received signals. The stored images are sent to the personal computer 420 via the communicating cable 412 after the observation. The personal computer 420 processes the images and outputs a video signal. The video signals are sent to the display 430 via the video cable 421, and displayed on the display 431.

The capsule endoscope system may configure the frequency of the carrier wave after the capsule endoscope 100 is powered. Therefore, the capsule endoscope 100 need not be configured to have the frequency of the carrier wave for each destination, so that the variety of capsule endoscopes 100 is reduced and manufacturing economy of scale is increased. Moreover, the capsule endoscope 100 need not comprise elements to obtain the frequency of the carrier wave from outside, so that it may be small and cost less to manufacture.

Note that the tip capacitor 212 may be a variable capacitor.

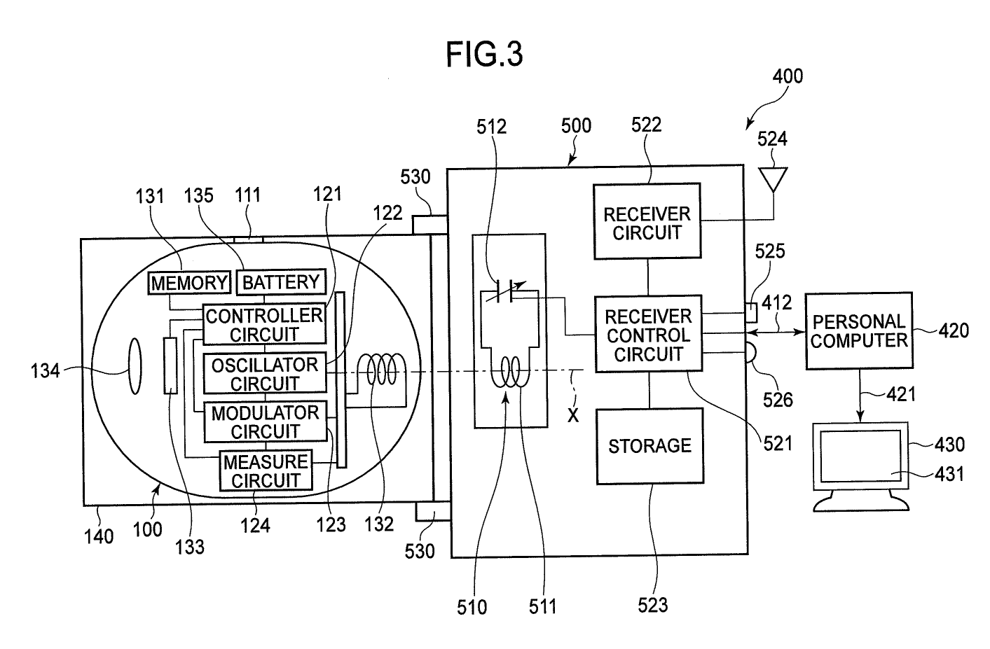

The capsule endoscope system 400 according to the second embodiment of the present invention is described below with reference to FIG. 3. The constructions similar to the first embodiment are given the same numbers and the descriptions are omitted.

The capsule endoscope system 400 mainly comprises a capsule endoscope 100, a second case 140 which stores the capsule endoscope 100, a second receiver 500, a personal computer 420, and display 430.

The second case 140 limits the movement of the capsule endoscope 100 linearly and rotationally about its axial direction. In other words, the capsule endoscope 100 may turn in along its circumference.

The second receiver 500 comprises a second resonator circuit 510, a receiver controller circuit 521 which controls the resonance frequency of the second resonator circuit 510, a receiver circuit 522, and a storage 523. The second resonator circuit 510 mainly comprises the variable capacitor 512 and a third coil 511. The resonance frequency of the second resonator circuit 510 varies according to the capacitance of the variable capacitor 512. The receiver circuit 521 controls the resonance frequency by changing the capacitance of the variable capacitor 512. The receiver circuit 522 receives a radio signal with the second antenna 524, and sends it to the receiver control circuit 521. The receiver control circuit 521 demodulates the radio signal, stores the modulated signal as images, and sends the images to the personal computer 420 via communication cable 412 after observation.

The second receiver 500 comprises a retainer 530. The retainer 530 keeps the second case 140 which stores the capsule endoscope 100 on the outer surface of the second receiver 500 so that the center axis X of the capsule endoscope 100 is substantially coaxial with the center axis of the third coil 511. As mentioned above, the center axis X of the capsule endoscope 100 is substantially coaxial with the center axis of the first coil 132. Therefore, the center axis of the first coil 132 is substantially coaxial with the third coil 511 thereof, even if the capsule endoscope rotates in the second case 140 during transport. The descriptions of the personal computer 420 and the display 430 are omitted because their constructions are similar to those of the first embodiment.

The first available frequency notification process which searches for an available frequency and notifies of an available frequency to the capsule endoscope 100 is described below.

The first available frequency notification process is executed when the user pushes the operating switch 525. The receiver control circuit 521 sends a command to search for an available frequency to the receiver circuit 522 when the receiver control circuit 521 detects that the operating switch 525 has been pushed. The receiver circuit 522 searches for an unused frequency, i.e., an available frequency within a predetermined frequency range. After that, the receiver circuit 522 sends the available frequency to the receiver control circuit 521. The receiver control circuit 521 adjusts the capacitance of the variable capacitor 512 so that the resonance frequency of the second resonator circuit 510 closely approximates the available frequency. The available frequency is stored in storage 523. The receiver control circuit 521 notifies the user of the completion of the adjustment by lighting the notification light 526 when the adjustment of the resonance frequency of the second resonator circuit 510 is completed. The first available frequency notification process thus ends.

The user pushes the power switch 111 through the second case 140 after confirming completion of the adjustment so that the capsule endoscope 100 is powered on. Then, the control circuit 121 adjusts the frequency of the radio signal by executing the frequency-decision process. The second receiver 500 receives a radio signal from the capsule endoscope 100 at the stored available frequency.

The adjusted frequency may deviate from the desired frequency due to temperature and humidity in use, and individual differences in the capsule endoscope 100. Therefore, the error-correction process described below is executed.

The receiver circuit 522 measures the carrier wave frequency of the radio signal sent by the capsule endoscope 100 when the second receiver 500 receives radio signal from the capsule endoscope 100. The receiver control circuit 521 adjusts the capacitance of the variable capacitor 512 when the measured frequency differs from the adjusted frequency. The capsule endoscope 100 adjusts the radio frequency again by the means described in the first embodiment so that the radio frequency is tuned to the target frequency. The error-correction process ends herewith. The error-correction process thus tunes the radio frequency to the target frequency.

The user takes out the capsule endoscope 100 from the second case 140, and swallows it. Thus begins the observation.

In the second embodiment, the radio frequency may be set at the last minute, and manufacturing economy of scale of the capsule endoscope system 400 is increased. Error between the radio frequency used by the second receiver 500 and the radio frequency used by the capsule endoscope 100 is eliminated. The radio frequency is configured according usage location so that the radio signal will not interfere with other electronic devices, will not be interfered with by other electronic devices, and will not be influenced by electromagnetic noises from nearby devices.

Note that resonance frequency may be configured by adjusting the inductance of the second coil 211 and the third coil 511.

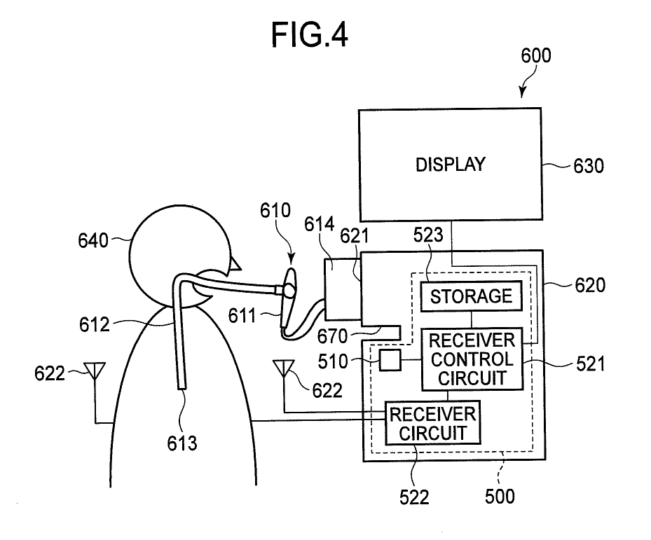

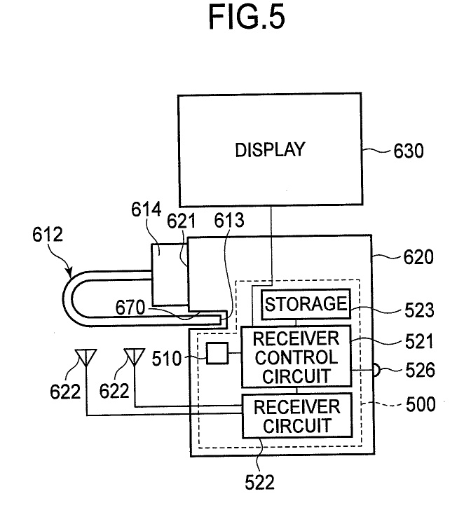

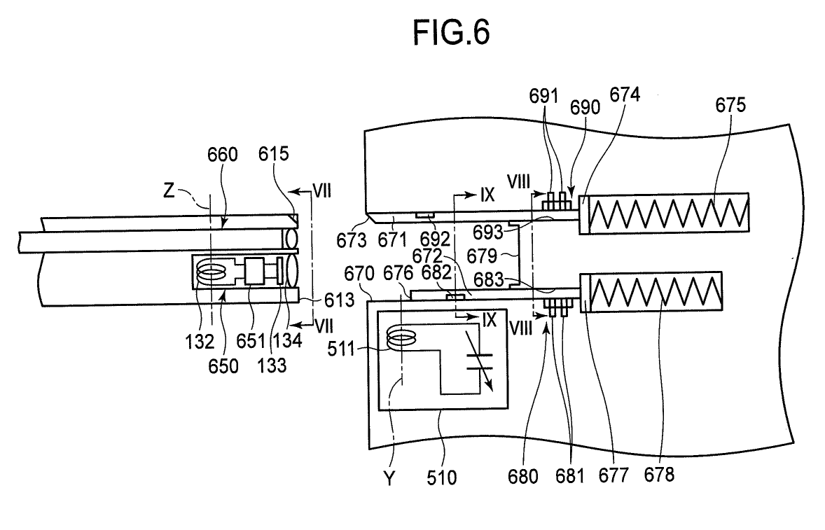

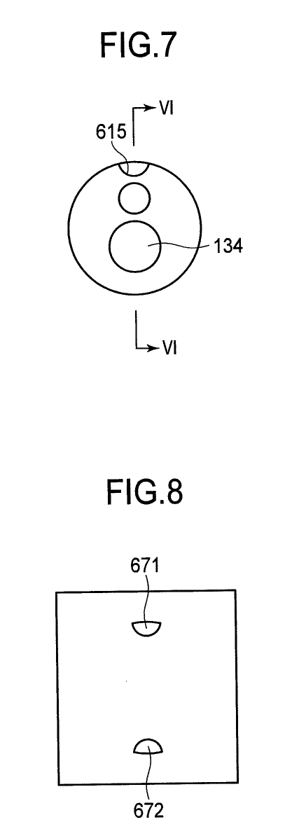

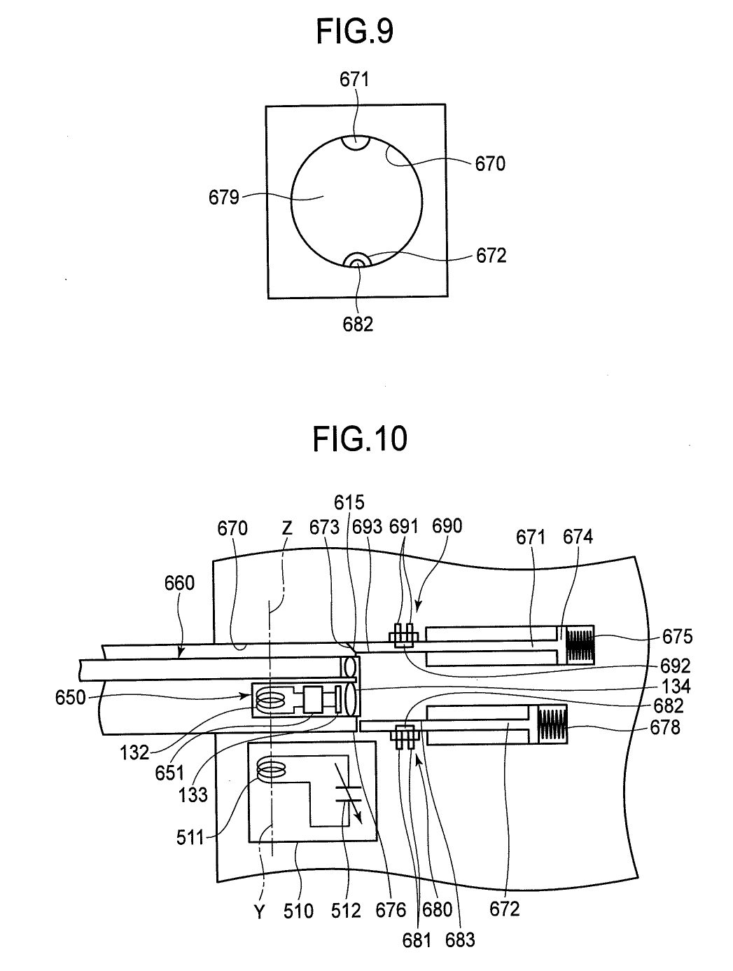

The first endoscope 610 and the capsule endoscope system 600 according to the third embodiment of the present invention are described below with reference to FIGS. 4-10. The constructions similar to those of the first and second embodiments are given the same numbers and the descriptions are omitted.

The endoscope system 600 mainly comprises the first endoscope 610 which takes images of observation subjects, an endoscope processor 620 which is connected with the proximal end of the first endoscope 610, and a display which is connected to the endoscope processor 620 and displays images.

The first endoscope 610 has an operating device 611 which is used for operating the first endoscope 610 and a flexible tube 612 which extends from the operating device. The user grabs the operating device 611 and operates the first endoscope 610. The flexible tube has a long cylindrical shape. The distal end of the first endoscope 610 has a first radio observation device 650 and a light optical system 660, and is inserted into the body 640 to be tested. The first radio observation device is detachable at the distal end 613.

A cutout 615 is provided on the circumference of the edge face of the distal end 613. The shape of the cutout 615 is a part of a lateral surface of a circular cone. On the cross-sectional surface traversing the center axis of the endoscope 610 and the center axis of the circular cone, the cutout 615 forms a line which draws closer to the lateral surface of the first endoscope 610 from the edge face of the distal end 613 to the proximal end 614. On the cross-sectional surface parallel to the edge face of the distal end 613, the cutout 615 forms substantially half of a circular arc.

The first radio observation device 650 mainly comprises a first controller 651 which controls the first radio observation device 650, the first coil 132 which functions as a transmitting antenna, the CCD 133 which takes images, and the lens 134 which is exposed on the edge face of the distal end 613.

The first controller 651 mainly comprises the control circuit, the resonator circuit, the demodulator circuit, the measuring circuit, the memory which stores information, and the battery which supplies electrical power to the elements of the first radio observation device 650. The constructions of the control circuit, the resonator circuit, the demodulator circuit, the measuring circuit, the memory, and the battery are similar to those of the first embodiment, so the descriptions are omitted.

The light optical system 660 projects light emitted from a light source (not shown) which is provided in the endoscope processor 620 to the observation subjects in the body to be tested. The projected light is reflected by the observation subject. A part of the reflected light enters an observation optical system provided in the first radio observation device 650.

The endoscope processor 620 mainly comprises a connector 621 which is connected with the proximal end of the first endoscope 610, an opening 670 which may accept the distal end of the first endoscope 610, the second receiving antennas 622 which receive signals sent by the first radio observation device 650, and the second receiver 500 which controls the communicating frequency of the first radio observation device 650.

The opening 670 has a cylindrical shape. The axial length of the opening 670 is longer than the distance between the distal end 613 and the first coil 132. On the internal surface of the opening 670, a first projection 671 which sets the circumferential position of the first endoscope 610, and the second projection 672 which works as a switch to start adjusting radio frequency.

The first projection 671 has a bar shape which extends from the back of the opening 670 to the open end of the opening 670, and has a substantially semicircular cross-sectional surface orthogonal to the longer direction. The first apex 673 which is proximal to the open end of the opening 670 has a part of a circular cone cut by a cylindrical surface which is parallel to the axis of the circular cone, and a shape corresponding to the cutout 615. The other end of the first projection 671 has a first spring receiver 674. The first spring receiver 674 engages a spring 675 provided in the endoscope processor 620, so that biases the first projection 671 toward the open end of the opening 670. A first guide 693 is provided in the endoscope processor 620. On the cross-sectional surface orthogonal to the longer direction of the opening 670, the cross-sectional surface of the first guide 693 is slightly larger that that of the first projection 671. The exterior surface of the first projection 671 may slide against the interior surface of the first guide 693.

A first contact 692 is provided on the first projection 671 near the open end of the opening 670. A first switch 690 has two first electrodes 691, and is provided on the interior surface of the first guide 693. The first switch 690 sends a signal to the receiver control circuit 521. The signal indicates whether two first electrodes 691 are electrically connected or not, i.e., whether the first switch 690 is on or off. When the first projection 671 is pushed by the distal end 613 into the back 679 of the opening 670, the first contact 692 electrically connects two first electrodes 691. Thereby, the first switch 690 is switched on.

The second projection 672 has a elongated projection which extends from the bottom of the opening 670 to a location near the open end of the opening 670, and is substantially semicircular on the cross-sectional surface orthogonal to the longer direction. The second apex 676 which is proximal to the open end of the opening 670 has a flat surface orthogonal to its longitudinal direction and engages the edge of the distal end 613. The other end of the second projection 672 has a second spring receiver 677. The second spring receiver 677 engages a spring 678 provided in the endoscope processor 620, thus biasing the second projection 672 toward the open end of the opening 670. A second guide 683 is provided in the endoscope processor 620. The radial cross-sectional area of the second guide 683 is slightly larger than that of the second projection 672 which fits into the second guide 683. The exterior surface of the second projection 672 may slide against interior surface of the second guide 683.

A second contact 682 is provided on the second projection 672 near the open end of the opening 670. A second switch 680 has two second electrodes 681, and is provided on the interior surface of the second guide 683. The second switch 680 send a signal to the receiver control circuit 521. The signal indicates whether two second electrodes 681 are electrically connected or not, i.e., whether the second switch 680 is turned on or off. When the second projection 672 is pushed by the distal end 613 into the back 679 of the opening 670, the second contact 682 electrically connects two second electrodes 681. Thus, the second switch 680 is switched on.

Note that the electrodes are provided in the first projection 671 or its perimeter. The second receiving antennas 622 may be directional or non-directional.

The construction of the second receiver 500 is the same as the one described in the second embodiment. The second resonator circuit 510 which is provided in the second receiver 500 is placed such that the center axis Z of the first coil 312 is substantially coaxial with the center axis Y of the third coil 511 when the distal end 613 is inserted into the back 679. The number and the position of the receiving antennas 622 are decided according to the intensity of the radio signals emitted by the first radio observation device 650.

The first apex 673 engages the cutout 615 so that the distal end 613 does not rotate in the opening 670. Therefore, the user may make the center axis Z of the first coil 312 substantially coaxial with the center axis Y of the third coil 511 by only inserting the distal end 613 into the back 679.

The observation of the body 640 with the endoscope system 600 is described below. The endoscope system 600 is constructed so as to observe the body 640 after tuning the communicating frequency of the first radio observation device 650. The description begins with the tuning of the communicating frequency of the first radio observation device 650.

The first radio observation device 650 comprises a switch (not shown), and is powered by operating the switch. The switch is operated by attaching the first radio observation device 650 into the distal end 613. The first radio observation device 650 executes the frequency-decision process after being powered. At this time, however, the frequency-decision process is not completed by the first radio observation device 650, because the receiver control circuit 521 has not completed the frequency control process as described later. Therefore, the following process is executed.

The cutout 615 engages the first projection 671 when the user inserts the distal end 613 into the opening 670. The user further inserts the distal end 613 so that the first projection 671 proceeds toward the back 671. The first spring receiver 674 compresses the spring 675, and then the distal end 613 engages the second projection 672. The distal end 613 is further inserted so that the first projection 671 and the second projection 672 proceed toward the back 671. The second spring receiver 677 compresses the spring 678, and then the distal end 613 engages the back 679.

The center axis Z of the first coil 132 is substantially coaxial with the center axis Y of the third coil 511 when the distal end 613 is placed in such position. The first contact 692 electrically connects two first electrodes 691 so that the first switch 690 is ON, and the second contact 682 electrically connects two second electrodes 681 so that the second switch 680 is ON.

The receiver control circuit 521 executes the frequency control process as described later when both the first switch 690 and the second switch 680 are ON.

Otherwise, in the case that ether the first switch 690 or the second switch 680 is ON, the receiver control circuit 521 judges that the distal end 613 is not properly inserted into the opening 670 and does not execute the frequency control process. In the case that both the first switch 690 and the second switch 680 are not ON, the receiver control circuit 521 judges that the distal end 613 is not inserted into the opening 670 and does not execute the frequency control process.

In the frequency control process, the receiver circuit 522 searches for unused frequencies, i.e., available frequencies among a certain frequency range, with the second receiving antenna 524. After that, the receiver circuit 522 sends available frequencies to the receiver control circuit 521. The receiver control circuit 521 selects a communicating frequency from available frequencies for communicating with the first radio observing device 650, and adjusts the capacitance of the variable capacitor 512 so that the resonance frequency of the second resonator circuit 510 closely approximates the available frequency. The communicating frequency is stored in storage 523 by the receiver control circuit 521.

The first radio observation device 650 adjusts the radio frequency by executing the frequency-decision process. Thus ends the frequency control process. Next, the first radio observation device 650 executes the feedback process as described hereafter.

The first controller 651 transmits a test signal using the DIP frequency stored in the memory 131 through the first coil 132. This transmission is executed without the CCD 133 so that the test signal will not include baseband signals. Thus, the test signal is easily created.

The receiver circuit 522 searches for the frequency of the test signal among a certain frequency range, and sends the detected frequency to the receiver control circuit 521. The receiver control circuit 521 notifies the user of the completion of adjustment by lighting the notification light 526 when the test signal frequency is in the acceptable range.

If the frequency of the test signal is not in the acceptable range, the receiving control circuit 521 executes the process described below. When the searched frequency is lower than the communicating frequency, the variable capacitor 512 is adjusted so that the resonance frequency of the resonator circuit 510 is slightly higher than its current frequency. The first controller 651 provided in the radio observation device 650 raises the frequency of the test signal according to the resonance frequency of the resonator circuit 510. Otherwise, the detected frequency is higher than the communicating frequency; the variable capacitor 512 is adjusted so that the resonance frequency of the resonator circuit 510 is slightly lower than its current frequency. The first controller 651 decreases the frequency of the test signal according to the resonance frequency of the resonator circuit 510. The frequency of the test signal approaches the communicating frequency by repeating these processes, and when it is in the acceptable range of the communicating frequency, the receiver control circuit 521 notifies the user of the completion of the adjustment by lighting the notification light 526. Thus ends the feedback process.

By carrying out the feedback process, the errors created between the communicating frequency and the available frequency and the individual difference of the first radio observation device 650 can be corrected.

The resonance frequency of the second resonator circuit 510 is adjusted without the user operating the operating switch 525.

The control circuit 121 starts imaging with the CCD 133, reads the DIP frequency from the memory 131, and sends the DIP frequency to the modulator circuit 123. The modulator circuit 123 receives the imaging signals from the control circuit 121, and modulates the imaging signals using the DIP frequency as a carrier wave. The radio image signals which are created by modulating the imaging signals are sent to the endoscope processor 620.

The receiver controller circuit 521 receives the radio image signals sent from the first radio observation device 650 using the communicating frequency stored in the storage 523. The received radio image signals are demodulated to yield the image signals and are sent to the endoscope processor 620. The display 630 displays images using the image signals sent by the endoscope processor 620. When the user swallows the distal end of the flexible tube 612, the first radio observation device 650 sends images of the body to be tested to the endoscope processor 620 as described above. By this means, the inner body may be observed.

According to this embodiment, the endoscope system 600 need not have signal wires for transmitting image signals from the CCD 133 to the endoscope processor 620 in the flexible tube 612. Therefore, the diameter of the flexible tube may be narrow.

Note that the first radio observation device need not be detachable, but may be integrally provided in the distal end 613.

The electrical power may be provided to the controller 651 through an electrical wire which is provided in the flexible tube 612 without the battery 135. The battery may be rechargeable.

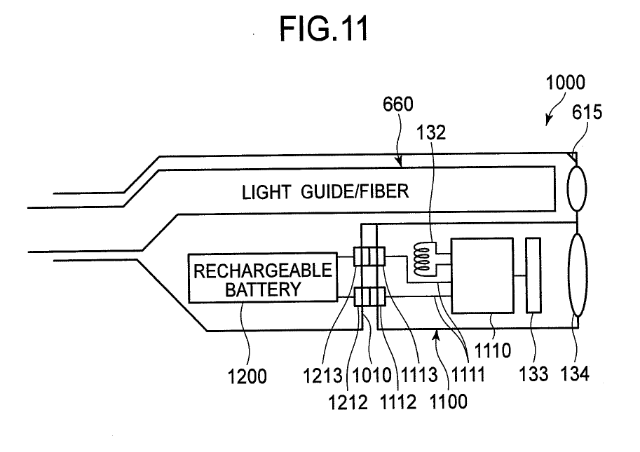

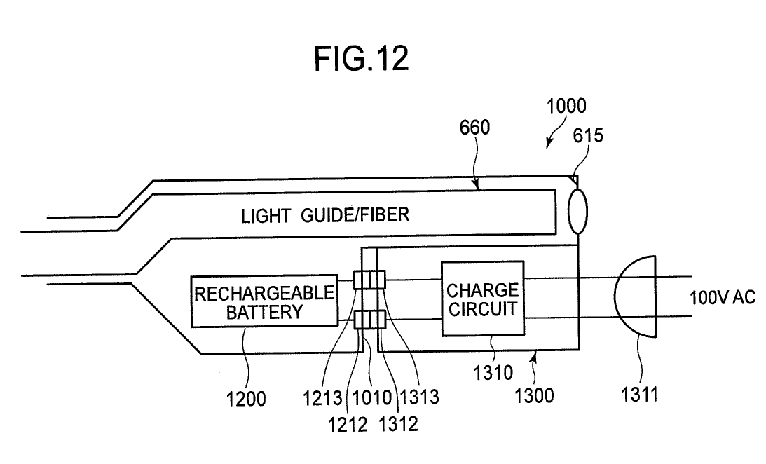

The endoscope system and the second endoscope 1000 according to the fourth embodiment of the present invention are described below with reference to FIGS. 11-12. The constructions similar to the first, second, and third embodiments are given the same numbers and the descriptions are omitted.

The endoscope system according to the present embodiment comprises the second endoscope 1000 which takes images of observation subjects, an endoscope processor which is connected with the proximal end of the second endoscope 1000, and a display which is connected to the endoscope processor and displays images taken by the second endoscope 1000. The constructions of the endoscope processor and the display are similar to the third embodiment, so that the descriptions are omitted.

The second endoscope 1000 comprises a second radio observation device 1100 which is detachable at its distal end and the rechargeable battery 1200 which is provided at the distal end. The distal end comprises a container portion 1010 of a size that can store the second radio observation device 1100. The first positive electrode 1212 and the second negative electrode 1213 are provided on the surface of the container portion 1010. The first positive electrode 1212 and the second negative electrode 1213 are connected to the rechargeable battery through wires.

The second endoscope 1000 mainly comprises a second controller 1110 which controls the second endoscope 1000, a first coil 132, a CCD 133, and a lens 134. The constructions of the first coil 132, the CCD 133, and the lens 134 are similar to those of the first embodiment, so the descriptions are omitted.

The second controller 1110 comprises a control circuit, a resonator circuit, a demodulator circuit, a measuring circuit, and a memory which stores information, but does not comprise a battery. The constructions of the control circuit, the resonator circuit, the demodulator circuit, the measuring circuit, and the memory are similar to those of the first embodiment, so the descriptions are omitted.

Electrical power is provided to the control circuit, the resonator circuit, the demodulator circuit, the measuring circuit, and the memory through two wires 1111. The two wires 1111 are respectively connected to the second positive electrode 1112 and the second negative electrode 1113 which are provided on the outer surface of the second radio observation device 1100.

When the second radio observation device 1100 is stored in the container portion 1010, the first positive electrode 1212 is connected to the second positive electrode 1112, and the first positive electrode 1213 is connected to the second negative electrode 1113. The second controller 1110 is electrically connected to the rechargeable battery 1200 herewith, and the second controller 1110 is powered by the rechargeable battery 1200.

The endoscope system according to the present embodiment comprises a battery charger 1300 which may charge the rechargeable battery 1200.

The battery charger 1300 comprises a charge circuit 1310 which controls the charging voltage, charging current, and charging time, a plug 1311 which supplies electrical power to the charge circuit 1310, and the third positive electrode 1312 and third negative electrode 1313 which are provided on the outer surface of the battery charger 1300.

The plug 1311 receives alternating power from a commercial power source and supplies it to the charge circuit 1310. The charge circuit 1310 rectifies the alternating power and outputs electrical power at a voltage and current suitable for recharging to the third positive electrode 1312 and the third negative electrode 1313.

When the battery charger 1300 is connected to the container portion 1010, the first positive electrode 1212 is connected to the third positive electrode 1312, and the first positive electrode 1213 is connected to the third negative electrode 1313. The charge controller 1310 is electrically connected to the rechargeable battery 1200, and the rechargeable battery 1200 is charged.

The observation procedure using the second endoscope 1000 is similar to the third embodiment, so that the descriptions are omitted.

Note that insulating materials which are provided to each positive electrode and negative electrode are omitted in the figures.

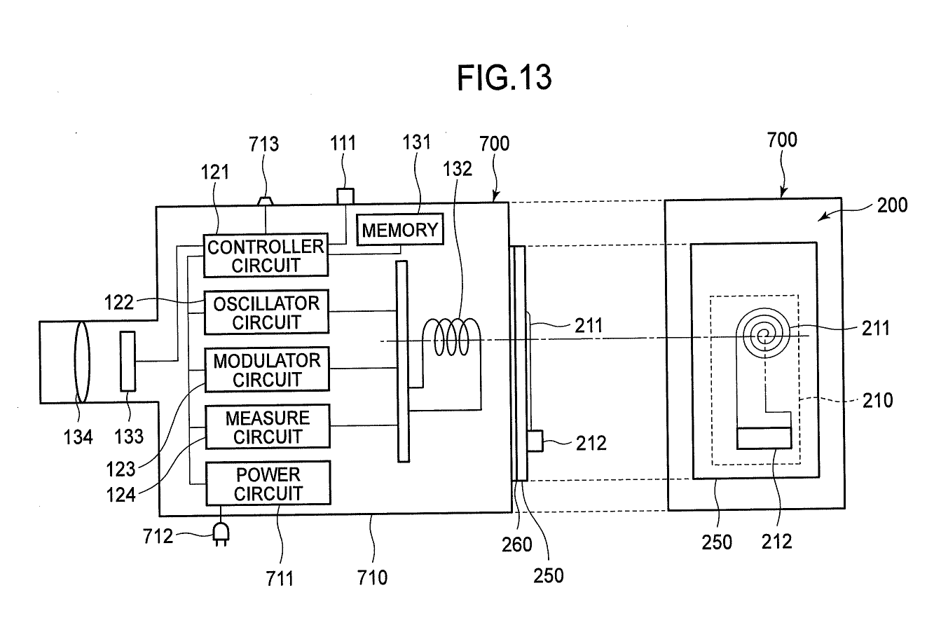

A first surveillance system and a first surveillance camera according to the fifth embodiment of the present invention are described below with reference to FIG. 13. The constructions similar to those of the first to fourth embodiments are given the same numbers and the descriptions are omitted.

The first surveillance system mainly comprises a first surveillance camera 700, a first receiver 410 which has a first receiver antenna 411, a personal computer 420, and a display 430.

The first surveillance camera 700 mainly comprises a power switch 111 which is used for turning on the first surveillance camera 700, the control circuit 121, the oscillator circuit 122, the modulator circuit 123, the measure circuit 124, the memory 131, the first coil 132, the CCD 133, the lens 134, a power circuit 711, and a notification light 713.

The power circuit receives alternating power from a commercial power source from the circuit 1310, rectifies and regulates it and supplies it to the components of the first surveillance camera 700. A chassis 710 of the first surveillance camera 700 has a lens tube which stores the lens 134 and the CCD 133, and a main body which stores the first coil and the circuits, e.g., the control circuit 121. The lens tube is connected to the front surface of the main body. The first coil 132 is provided in the chassis 710 near the back surface which is behind the front surface. The memory 131 stores the control program of the first surveillance camera 700 and the transmission frequency (the DIP frequency). When the first surveillance camera 700 is shipped, the value of the DIP frequency indicates that the DIP frequency is not set. The notification light 713 is connected to the control circuit 121 and notifies the operating state of the first surveillance camera 700 to the user by a light signal.

The resonance circuit plate 250 is attached to the chassis 710 with an adhesive sheet, so that the center axis of the second coil 211 is substantially coaxial with the center axis of the first coil 132 and the distance in the axial direction between the first coil 132 and the second coil 211 is in a range that electromagnetic induction occurs between the first coil 132 and the second coil 211.

The first receiver 410 is provided apart from the first surveillance camera 700. The distance between the first surveillance camera 700 and the first receiver 410 is such that the first receiver 410 may reliably receive the radio signal emitted by the first surveillance camera 700. The first receiver 410 demodulates the radio signals which are received through the first receiving antenna 411, converts the demodulated signals to images, and sends the images to the personal computer 420 through the communication cable 421. The personal computer processes the received images and outputs the video signals to the display 431 through the video cable 421. The display 431 receives the video signals and displays them.

The procedure for using the first surveillance camera 700 is described below.

The user pushes the power switch 111 so that the first surveillance camera 700 is powered on. Then, the control circuit 121 reads the control program from the memory 131, and begins controlling the components of the first surveillance camera 700.

At that time, if the value of the DIP frequency indicates that the DIP frequency has not yet been set, the control circuit 121 executes the frequency-decision process as described hereinbefore.

When the frequency-decision process ends, the control circuit 121 lights the notification light 713 and notifies the user of the completion of the process. The user recognizes this notification, pushes the power switch 111 so that the power of the first surveillance camera 700 is turned off, and then may store the camera.

When the user powers on the first surveillance camera 700 by pushing the power switch 111, the control circuit 121 reads the control program and the DIP frequency from the memory 131. The DIP frequency stored in the memory 131 does not indicate that the DIP frequency is not set, so that the controller 121 begins imaging with the CCD 133. The image signal output by the CCD 133 is processed by the control circuit 121. The modulator circuit 123 modulates the processed signal as a carrier wave. The modulated signal is transmitted to the outside of the first surveillance camera 700 through the first coil 132. The first receiver 410 receives the radio signal.

According to this embodiment, the first surveillance camera 700 configures the frequency of the carrier wave after its shipment. Therefore, the plurality of first surveillance cameras 700 need not be configured to have the carrier wave frequency particular to each destination, so the number of variants of the first surveillance cameras 700 may be reduced and manufacturing economy of scale is increased. Moreover, the first surveillance cameras 700 need not comprise elements to obtain the frequency of the carrier wave from outside, so that it may be small and the cost of manufacturing will be decreased.

Note that a battery may be used instead of the power circuit 711.

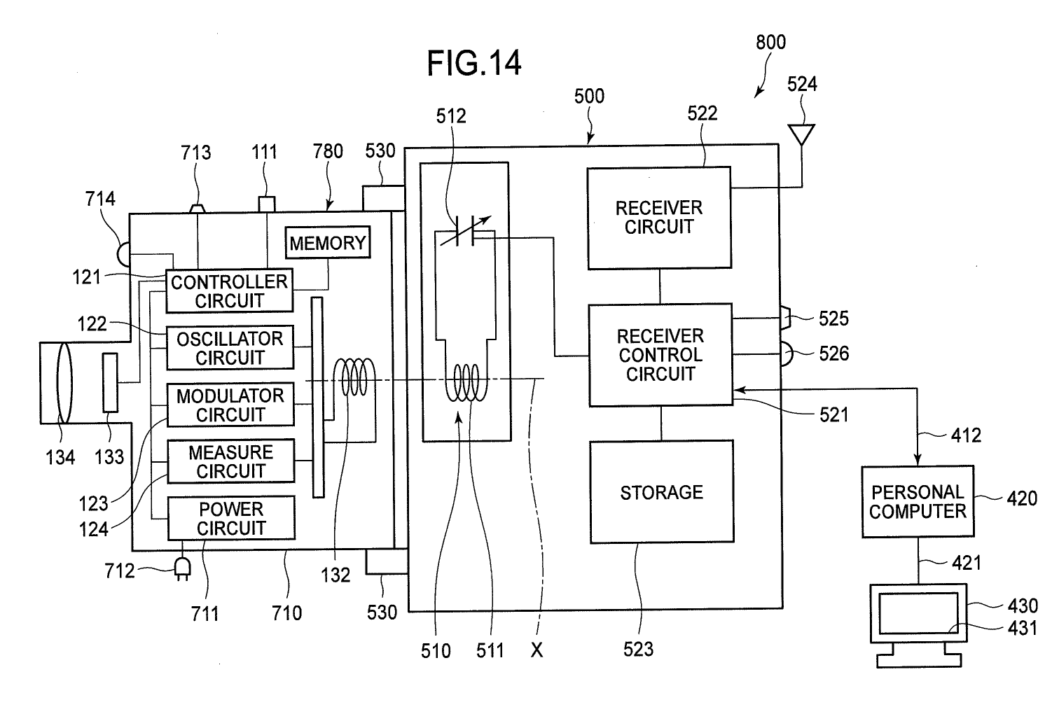

A second surveillance system 800 and a second surveillance camera 780 according to the sixth embodiment of the present invention are described below with reference to FIGS. 14-16. The constructions similar to the first to those of the fifth embodiment are given the same numbers and the descriptions are omitted.

The second surveillance system 800 mainly comprises a plurality of second surveillance cameras 780, a second receiver 500, the personal computer 420, and the display 430.

The second receiver 500 may communicate with the second surveillance cameras 780, numbering n-i, by radio waves. The storage 523 stores the DIP frequency which is allocated to each second surveillance camera 780 and the number of the allocated DIP frequency. n is an integer greater than or equal to 2.

The second surveillance camera 780 comprises a microphone 714 which is connected to the control circuit 121. The microphone 714 converts sound made by the observation subjects into an electrical signal and sends it to the control circuit 121.

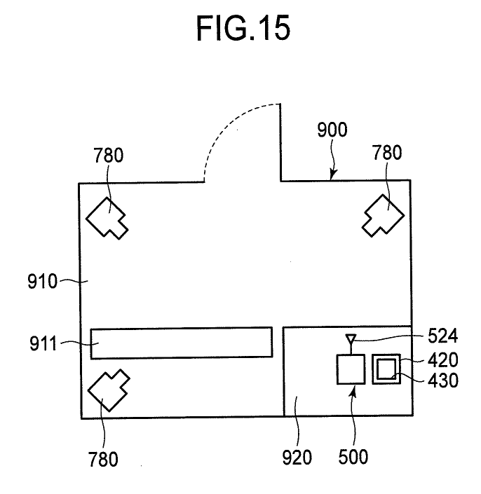

The positioned second surveillance cameras 780 are shown in FIG. 15. The plurality of second surveillance cameras 780 are attached to the ceiling of a bank's front counter room 910. A counter 911 is provided in the front counter room 910. A surveillance room 920 is provided in the neighborhood of the counter 911. The second receiver 500, the personal computer 420, and the display 430 are provided in the surveillance room 920.

Each of the second surveillance cameras 780 has a different DIP frequency, and sends radio signals to the second receiver 500 using its own DIP frequency.

The distance between the second surveillance camera 780 and the second receiver 500 is such that the second receiver 500 may reliably receive the radio signal emitted by the second surveillance cameras 780. The second receiver 500 demodulates the radio signals which are received through the second receiving antenna 524, converts the demodulated signals to images, and sends the images to the personal computer 420 through the communication cable 421. The personal computer processes the received images and outputs the video signals to the display 431 through the video cable 421. The display 431 receives the video signals and displays them.

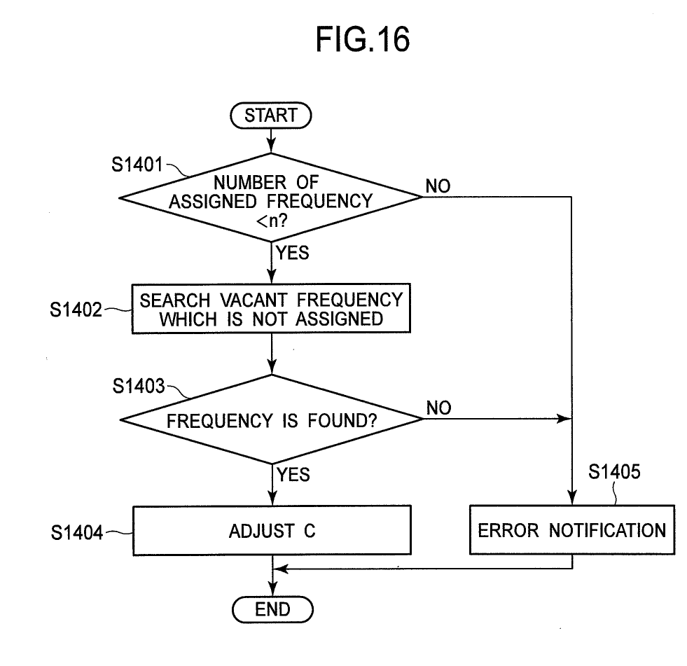

A second available frequency notification process which is executed for searching available frequencies and notifying the second surveillance camera 780 of the available frequency is described below with reference to FIG. 16. The second available frequency notification process differs from the first available frequency notification process in that the second available frequency notification process may adjust the frequencies of the plurality of the camera.

Once the retainer 530 is holding the second surveillance camera 780 on the outer surface of the second receiver 500, the user pushes the operating switch 525. The receiver controller 521 detects that the operating switch 525 is pushed, and executes the second available frequency notification process.

In step S1401, the receiver controller 521 determines whether the number of the DIP frequency assigned to the second surveillance camera 780 is less than n or not. In the case it is less than n, the process proceeds to step S1402. In the case it is greater than or equal to n, the process proceeds to step S1405, and notifies the user by blinking the notification light 526 or sounding a notification buzzer (not shown) to indicate that a usable DIP frequency was not found.

In step S1402, the receiver circuit 522 searches for an available frequency different from the assigned DIP frequency within a certain frequency range.

In step S1403, it is determined whether an available frequency has been found or not. If one is found, the process proceeds to step S1404. If one is not found, the process proceeds to step S1405, and notifies the user by flashing the notification light 526 or sounding a notification buzzer (not shown) to indicate that an available frequency was not found.

In step S1404, the receiver circuit 522 sends the available frequency to the receiver control circuit 521. The receiver control circuit 521 adjusts the capacitance of the variable capacitor 512 so that the resonance frequency of the second resonator circuit 510 closely approximates the available frequency. The available frequency is stored in storage 523. The receiver control circuit 521 notifies the user of the completion of the adjustment by lighting the notification light 526 when the adjustment of the resonance frequency of the second resonator circuit 510 is complete. Thus ends the second available frequency notification process.

Next, the user secures the second surveillance camera 780 onto the outer surface of the second receiver 500 with the retainer 530, and pushes the operating switch 525, to execute the second available frequency notification process again. Executing the second available frequency notification process for each second surveillance camera 780, the DIP frequency of each second surveillance camera 780 is configured.

According to the embodiment, the surveillance camera may be provided without wiring such as a wired surveillance camera would require, and configuring the wireless surveillance camera is simpler.

Note that in the second available frequency notification process, the second surveillance camera 780 may send a signal indicating the completion of the assignment of the available frequency to the second receiver 500 through the first coil 132. When the second receiver 500 receives this signal it notifies the user of the completion of assignment of an available frequency by blinking the notification light 526 or sounding a notification buzzer (not shown). The second surveillance camera 780 need not include the notification light 713. This makes the second surveillance camera 780 smaller and decreases its manufacturing cost.

Moreover, in the second available frequency notification process, when the available frequency is not assigned because the receiver controller circuit 521 is unable to adjust the resonance frequency of the second resonator circuit 510 to an available frequency, the second surveillance camera 780 may send a signal indicating the failure of available frequency assignment to the second receiver 500. Once it receives the signal, the second receiver 500 executes the second available frequency notification process again. Alternatively, the second receiver 500 may notify the user of the failure of available frequency assignment by blinking the notification light 526 or sounding a notification buzzer (not shown).

In this embodiment, the error-correction process may be executed.

Note that in all embodiments, rather than CCD 133, the image sensor may be any device that converts light to an electrical signal, e.g., a CMOS chip.

In all embodiments, the radio observation device may transmit signals through wired lines. For example, the second surveillance cameras 780 may be connected to the second receiver 500 through a coaxial cable and send signals multiplexed with carrier waves which are provided for each second surveillance cameras 780.

The present invention may be applied to a wireless microphone system by substituting the CCD 133 with a microphone and the display 430 with a speaker.

Although the embodiment of the present invention has been described herein with reference to the accompanying drawings, obviously many modifications and changes may be made by those skilled in the art without departing from the scope of the invention.

The present disclosure relates to subject matter contained in Japanese Patent Application No. 2007-305855 (filed on Nov. 27, 2007), which is expressly incorporated herein, by reference, in its entirety.

1. A radio observation device which transmits information on observation subjects comprising:
a first coil;
an oscillator that generates signals flowing through said first coil within a certain range of frequencies;
a first frequency controller that is connected to said oscillator, and adjusts the oscillating frequency of the signal flowing through said first coil by controlling said oscillator;
a frequency-measuring device that measures the resonance frequency of a signal flowing through said first coil and a signal flowing through a second coil being provided external to said radio observation device; and
a transmitter that transmits the information on the observation subjects through said first coil by a carrier wave which oscillates at the resonance frequency.

2. The radio observation device according to claim 1,
wherein said frequency measuring device measures current flowing through said first coil when said first frequency controller adjusts an oscillating frequency of said first coil, and determines that the resonance frequency is the oscillating frequency of the signal through said first coil when the current is minimal.

3. The radio observation device according to claim 1, further comprising a memory that stores the resonance frequency.

4. The radio observation system comprising:
a radio observation device which transmits information on observation subjects comprising a first coil, an oscillator that generates signals flowing through said first coil within a certain range of frequencies, a first frequency controller that is connected to said oscillator, and adjusts the oscillating frequency of the signal flowing through said first coil by controlling said oscillator, a frequency-measuring device that measures the resonance frequency of a signal flowing through said first coil and a signal flowing through a second coil being provided external to said radio observation device, and a transmitter that transmits the information on the observation subjects through said first coil by a carrier wave which oscillates at the resonance frequency; and,
a first resonator that has a first resonance frequency;
wherein signals flowing through said first coil resonate with said first resonator at the first resonance frequency so that said first frequency controller adjusts the oscillating frequency of signals flowing through said first coil.

5. The radio observation system comprising:
a data receiver having a second resonator that may change the resonance frequency, a second frequency controller that changes the resonance frequency by controlling said second resonator, and a measuring device that measures a first frequency which is suitable for radio transmission; and,
a radio observation device which transmits information on observation subjects comprising a first coil, an oscillator that generates signals flowing through said first coil within a certain range of frequencies, a first frequency controller that is connected to said oscillator, and adjusts the oscillating frequency of the signal flowing through said first coil by controlling said oscillator, a frequency-measuring device that measures the resonance frequency of a signal flowing through said first coil and a signal flowing through a second coil being provided external to said radio observation device, and a transmitter that transmits the information on the observation subjects through said first coil by a carrier wave which oscillates at the resonance frequency;
wherein said second frequency controller controls said second resonator so that said second resonator has a resonance frequency which is substantially the same as the first frequency.

6. The radio observation device according to claim 1, being a capsule-shaped medical device that is delivered into a body to be tested, and sends information by radio transmission.

7. The radio observation device according to claim 6,
wherein the information is image information.

8. A capsule-shaped medical device system comprising:
a capsule-shaped medical device which is delivered into a body to be tested, and sends information by radio transmission comprising a first coil, an oscillator that generates signals flowing through said first coil, within a certain range of frequencies, a first frequency controller that is connected to said oscillator, and adjusts the oscillating frequency of the signal flowing through said first coil by controlling said oscillator, a frequency-measuring device that measures the resonance frequency of a signal flowing through said first coil and a signal flowing through a second coil which is provided external to said radio observation device, and a transmitter that transmits the information on the observation subjects through said first coil by a carrier wave which oscillates at the resonance frequency;
a first resonator that has a first resonance frequency; and,
a case that stores said capsule-shaped medical device;
wherein said first resonator has said second coil, and is mounted on said case so that said first coil is adjacent to said second coil, and
wherein said first frequency controller adjusts the oscillating frequency of signals flowing through said first coil so that signals flowing through said first coil resonate with said first resonator at said first resonance frequency.

9. The capsule-shaped medical device system according to claim 8,
wherein said first resonator is mounted on said case so that an axis of said first coil and an axis of said second coil are coaxially arranged and the distance in the axial direction between said first coil and said second coil is in a range in which electromagnetic induction occurs between said first coil and said second coil.

10. The capsule-shaped medical device system according to claim 8 further comprising a power switch which is provided to be operable from the outside of said case, and aid first frequency controller adjusting the oscillating frequency of said first coil by controlling said oscillator when said power switch is operated so as to power the capsule-shaped medical device.

11. An endoscope comprising;
a radio observation device which transmits information on observation subjects comprising a first coil, an oscillator that generates signals flowing through said first coil, within a certain range of frequencies, a first frequency controller that is connected to said oscillator, and adjusts the oscillating frequency of the signal flowing through said first coil by controlling said oscillator, a frequency-measuring device that measures the resonance frequency of a signal flowing through said first coil and a signal flowing through a second coil which is provided external to said radio observation device, and a transmitter that transmits the information on the observation subjects through said first coil by a carrier wave which oscillates at the resonance frequency;
said radio observation device being provided at the distal end of said endoscope; and
the distal end of said endoscope being delivered into a body to be tested and sending information on the interior of the body by radio transmission.

12. An endoscope system comprising:
an endoscope comprising a radio observation device which transmits information on observation subjects comprising a first coil, an oscillator that generates signals flowing through said first coil, within a certain range of frequencies, a first frequency controller that is connected to said oscillator, and adjusts the oscillating frequency of the signal flowing through said first coil by controlling said oscillator, a frequency-measuring device that measures the resonance frequency of a signal flowing through said first coil and a signal flowing through a second coil which is provided external to said radio observation device, and a transmitter that transmits the information on the observation subjects through said first coil by a carrier wave which oscillates at the resonance frequency, said radio observation device being provided at the distal end of said endoscope, and the distal end of said endoscope being delivered into a body to be tested and sending information on the interior of the body by radio transmission;
an endoscope processor that has a opening that receives the distal end of the endoscope and is connected to the proximal end of the endoscope;
said endoscope processor having a first resonator which is provided around the opening and has a first resonance frequency;
said first resonator having said second coil which is provided in said endoscope processor so that said first coil is adjacent to said second coil; and
said first frequency controller adjusting the oscillating frequency of signals flowing through said first coil so that signals flowing through said first coil resonate with said first resonator at said first resonance frequency.

13. The endoscope system according to claim 12, wherein said endoscope processor comprises a detector which is provided in an inner portion of the opening and detects that said distal end is inserted into the opening.

14. The endoscope system according to claim 13 further comprising a power switch which is provided to be operable from the outside of said case, wherein said first frequency controller adjusts the oscillating frequency of said first coil by controlling said oscillator when said detector detects that said distal end is inserted into the opening.

15. The endoscope system according to claim 13 wherein said distal end has a flexible cylindrical shape, said opening has a cylindrical shape of which the radial length is slightly larger than that of said distal end, and said distal end is restricted to move in the circumferential direction of the opening when said distal end engages said detector.

16. The radio observation device according to claim 1 being a surveillance camera which is provided at a location for observing subjects and transmits images and/or sound from the observation subjects.
